(19) United States
(12) Patent Application Publication
(10) Pub. No.: US 2026/0146280 A1
(43) Pub. Date: May 28, 2026

(54) METHODS, COMPOSITIONS, AND KITS FOR DETERMINING THE LOCATION OF AN ANALYTE IN A BIOLOGICAL SAMPLE

(71) Applicant: 10x Genomics, Inc., Pleasanton, CA (US)

(72) Inventors: Malte Kuhnemund, Stockholm (SE); Christina Galonska, Stockholm (SE)

(21) Appl. No.: 19/121,955

(22) PCT Filed: Oct. 17, 2023
(86) PCT No.: PCT/US2023/035322

§ 371 Date: Apr. 17, 2025

Related U.S. Application Data
(60) Provisional application No. 63/416,751, filed on Oct. 17, 2022.

(51) Int. Cl. C12Q 1/6841; C12Q 1/34; C12Q 1/48; C12Q 1/6844; C12Q 1/6876

(52) U.S. Cl. CPC C12Q 1/6841; C12Q 1/34; C12Q 1/48; C12Q 1/6844; C12Q 1/6876; C12Y 207/07031; C12Y 207/07049; C12Y 306/00; C12Q 2600/16

(57) ABSTRACT

Provided herein are methods, compositions, and kits for the spatial analysis of target nucleic acids in biological samples by their 5′ end. An exemplary method includes: contacting a biological sample with a primer that hybridizes the target nucleic acid; hybridizing the primer to the target nucleic acid and extending using the target nucleic acid as a template to generate an extension product; incorporating a non-templated polynucleotide sequence including at least three nucleotides to the 3′ end of the extension product; hybridizing the polynucleotide sequence of the extension product to a capture domain on an array including a plurality of capture probes that include a spatial barcode and the capture domain; and determining the spatial barcode sequence, or complement thereof, and all or a portion of the extension product, or complement thereof, and using the determined sequences to determine the location of the target nucleic acid in the biological sample. The non-templated polynucleotide sequence may be C-C-C or C-G-C introduced by terminal transferase or may be introduced by SMART technology using reverse transcriptase.

### CROSS-REFERENCE TO RELATED APPLICATIONS

This application claims the benefit of priority to U.S. Provisional Ser. No. 63/416,751, filed on Oct. 17, 2022, the contents of which are incorporated herein by in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Sequencing nucleic acid libraries generated from spatial array analysis generally biases capture of the 3′ end of the captured analytes. However, 5′ end sequences can contain valuable information about the target analyte. Thus, strategies are needed to sequence regions more than about 1 kilobase away from the 3′ end of nucleic analytes, such as 5′ ends, in nucleic acid libraries generated from spatial array analyses.

### SUMMARY

The present disclosure features methods of capturing analytes on a spatial array, where the spatial array includes a plurality of capture probes and a capture probe of the plurality of capture probes includes a spatial barcode and a capture domain. Since capture on a spatial array generally biases 3′ end sequences of nucleic acid analytes, methods are needed to capture the 5′ end of the nucleic acid analyte, or a complement thereof. For example, nucleic acid analytes can be reverse transcribed with a primer including a sequence complementary to a target nucleic acid and an adapter (e.g., a sequencing adapter) to generate an RNA/DNA (e.g., cDNA duplex). The reverse transcriptase can add a non-templated polynucleotide sequence to the end of the cDNA which hybridizes to the capture domain of the capture probe. The target nucleic acid can also be digested resulting in a single-stranded product that is captured by a capture probe on the array.

Provided herein are methods for determining a location of a target nucleic acid in a biological sample, including: (a) contacting the biological sample with a primer including a nucleic acid sequence that hybridizes to a complementary sequence in the target nucleic acid and a functional domain; (b) hybridizing the primer to the target nucleic acid and extending the primer using the target nucleic acid as a template to generate an extension product; (c) incorporating a polynucleotide sequence including at least three nucleotides to the 3′ end of the extension product; (d) hybridizing the polynucleotide sequence of the extension product to a capture domain on an array, where the array comprise a plurality of capture probes, and where a capture probe of the plurality of capture probes includes a spatial barcode and the capture domain; and (e) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

In some embodiments, the biological sample is disposed on the array including the plurality of capture probes. In some embodiments, the biological sample is disposed on a substrate. In some embodiments, such substrate does not include the array comprising the plurality of capture probes. In some embodiments, the method includes aligning the substrate with the array, such that a least a portion of the biological sample is aligned with at least a portion of the array.

In some embodiments, the hybridizing in step (d) includes passive migration, e.g., of the extension product to the array. In some embodiments, the hybridizing in step (d) includes active migration e.g., of the extension product to the array, and optionally, where the active migration includes electrophoresis.

In some embodiments, the polynucleotide sequence in step (c) includes a homopolynucleotide sequence (e.g., 5′-CCC-3′). In some embodiments, the polynucleotide sequence in step (c) includes a heteropolynucleotide sequence (e.g., 5′-CGC-3′).

In some embodiments, the capture domain includes a fixed sequence.

In some embodiments, the primer includes a random sequence. In some embodiments, the random sequence includes a random hexamer. In some embodiments, the random sequence includes a random decamer. In some embodiments, the primer includes a homopolymer sequence. In some embodiments, the homopolymer sequence is a poly(T) sequence. In some embodiments, the primer includes a sequence complementary to a nucleic acid sequence encoding a constant region of an immune cell receptor (e.g., an antibody). In some embodiments, the primer includes a sequence complementary to a nucleic acid sequence encoding a constant region of a B cell receptor. In some embodiments, the primer includes a sequence complementary to a nucleic acid sequence encoding a constant region of a T cell receptor.

In some embodiments, the array includes one or more features.

In some embodiments, the functional domain includes a primer binding site or a sequencing specific site.

In some embodiments, the target nucleic acid is an RNA. In some embodiments, the RNA is mRNA. In some embodiments, the mRNA includes a sequence encoding an immune cell receptor. In some embodiments, the mRNA includes a sequence encoding a T cell receptor. In some embodiments, the mRNA includes a sequence encoding a B cell receptor.

In some embodiments, incorporating the polynucleotide sequence to the 3′ end of the extension product in step (c) includes use of a terminal deoxynucleotidyl transferase. In some embodiments, incorporating the polynucleotide sequence to the 3′ end of the extension product in step (c) includes use of a reverse transcriptase.

In some embodiments, the method includes removing the target nucleic acid, or any other nucleic acid hybridized to the extension product, before the polynucleotide sequence hybridizes to the capture domain of the capture probe on the array in step (d). In some embodiments, the removing includes use of an RNase. In some embodiments, the RNase is RNaseH. In some embodiments, the removing includes use of heat.

In some embodiments, the method includes fixing the biological sample. In some embodiments, fixing the biological sample includes the use of a fixative selected from the group consisting of: ethanol, methanol, acetone, formaldehyde, paraformaldehyde-Triton, glutaraldehyde, and combinations thereof.

In some embodiments, the method includes staining the biological sample. In some embodiments, the staining includes use of eosin and/or hematoxylin. In some embodiments, the staining includes the use of a detectable label selected from the group consisting of a radioisotope, a fluorophore, a chemiluminescent compound, a bioluminescent compound, and a combination thereof.

In some embodiments, the method includes imaging the biological sample.

In some embodiments, the method includes before (e), performing a step of extending a 3′ end of the extension product of step (d) using the capture probe as a template, thereby generating an extended capture product, and/or performing a step of extending a 3′ end of the capture probe using the extension product of step (d) as a template, thereby generating an extended capture probe.

In some embodiments, the extended capture product is removed from the capture probe on the array. In some embodiments, the removing includes use of heat or KOH.

In some embodiments, the method includes generating a sequence library. In some embodiments, the determining in step (e) includes sequencing. In some embodiments, the sequencing includes high-throughput sequencing.

In some embodiments, the method includes a step of permeabilizing the biological sample. In some embodiments, the permeabilizing includes use of an organic solvent, a detergent, an enzyme, or a combination thereof. In some embodiments, the permeabilizing includes use of an endopeptidase, a protease sodium dodecyl sulfate, polyethylene glycol tert-octylphenyl ether, polysorbate 80, polysorbate 20, N-lauroyIsarcosine sodium salt solution, saponin, Triton X-100™, Tween-20™, or combinations thereof. In some embodiments, the endopeptidase is pepsin or proteinase K.

In some embodiments, the capture probe includes one or more functional domains, a unique molecular identifier, a cleavage domain, or combinations thereof.

In some embodiments, the biological sample is a tissue sample. In some embodiments, the tissue sample is a fixed tissue sample. In some embodiments, the fixed tissue sample is a formalin-fixed paraffin embedded tissue sample, a paraformaldehyde-fixed tissue sample, a methanol-fixed tissue sample, or an acetone-fixed tissue sample. In some embodiments, the tissue sample is a fresh frozen tissue sample. In some embodiments, the biological sample is a tissue section. In some embodiments, the tissue section is a fixed tissue section. In some embodiments, the fixed tissue section is a formalin-fixed paraffin embedded tissue section, a paraformaldehyde-fixed tissue section, a methanol-fixed tissue section, or an acetone-fixed tissue section.

In some embodiments, step (b) includes generating a plurality of extension products using a plurality of primers. In some embodiments, two or more primers of the plurality of primers hybridize to different sequences in the target nucleic acid. In some embodiments, extending the primer to generate an extension product in step (b) includes use of a reverse transcriptase, optionally where the reverse transcriptase has strand displacement activity.

In some embodiments, the target nucleic acid encodes V and J sequences of an immune cell receptor. In some embodiments, the target nucleic acid encodes V, D, and J sequences of an immune cell receptor.

Also provided herein are methods for determining a location of a target nucleic acid in a biological sample, including: (a) contacting the biological sample with a plurality of primers, where the plurality of primers collectively comprise nucleic acid sequences that hybridize to complementary sequences in the target nucleic acid and a functional domain; (b) hybridizing the plurality of primers to the target nucleic acid and extending one or more primers of the plurality of primers using the target nucleic acid as a template to generate one or more extension products; (c) incorporating a polynucleotide sequence to the 3′ end of the one or more extension products; (d) hybridizing the polynucleotide sequence of the one or more extension products of (c) to a plurality of capture probes on an array, where each capture probe in the plurality of capture probes comprises a spatial barcode and a capture domain; and for an extension product in the one or more extension products, (e) determining (i) the sequence of the spatial barcode of the capture probe to which the extension product is hybridized, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

In some embodiments, the biological sample is disposed on the array including the plurality of capture probes. In some embodiments, such substrate does not include the array comprising the plurality of capture probes. In some embodiments, the biological sample is disposed on a substrate. In some embodiments, the method includes aligning the substrate with the array, such that at least a portion of the biological sample is aligned with at least a portion of the array.

In some embodiments, the hybridizing in step (d) is facilitated by passive migration, e.g., of the one or more extension products to the array. In some embodiments, the hybridizing in step (d) is facilitated by active migration, e.g., of the one or more extension products to the array, and optionally, where the active migration includes electrophoresis.

In some embodiments, the polynucleotide sequence in step (c) includes a homopolynucleotide sequence (e.g., 5′-CCC-3′). In some embodiments, the polynucleotide sequence in step (c) includes a heteropolynucleotide sequence (e.g., 5′-CGC-3′).

In some embodiments, the capture domain includes a fixed sequence.

In some embodiments, a primer in the plurality of primers includes a random sequence. In some embodiments, the random sequence includes a random hexamer. In some embodiments, the random sequence includes a random decamer. In some embodiments, the plurality of primers includes a sequence complementary to a nucleic acid sequence encoding a constant region of immune cell receptor (e.g., an antibody). In some embodiments, a primer in the plurality of primers includes a sequence complementary to a nucleic acid sequence encoding a constant region of a B cell receptor. In some embodiments, a primer in the plurality of primers includes a sequence complementary to a nucleic acid sequence encoding a constant region of a T cell receptor. In some embodiments, a primer in the plurality of primers includes a homopolymer sequence. In some embodiments, the homopolymer sequence includes a poly(T) sequence.

In some embodiments, the array includes one or more features.

In some embodiments, the functional domain includes a primer binding site or a sequencing specific site.

In some embodiments, the target nucleic acid is an RNA. In some embodiments, the RNA is mRNA. In some embodiments, the mRNA includes a sequence encoding a T cell receptor. In some embodiments, the mRNA includes a sequence encoding a B cell receptor. In some embodiments, incorporating the polynucleotide sequence to the 3′ end of the extension product in step (c) includes use of a reverse transcriptase. In some embodiments, incorporating the polynucleotide sequence to the 3′ end of the extension product in step (c) includes use of a terminal deoxynucleotidyl transferase.

In some embodiments, the method includes removing the target nucleic acid, or any other nucleic acid hybridized, to the one or more extension products before the polynucleotide sequence hybridizes to the plurality of capture probes on the array. In some embodiments, the removing includes use of an RNase. In some embodiments, the RNase is RNaseH. In some embodiments, the removing includes use of heat.

In some embodiments, the method includes fixing the biological sample. In some embodiments, fixing the biological sample includes the use of a fixative selected from the group consisting of: ethanol, methanol, acetone, formaldehyde, paraformaldehyde-Triton, glutaraldehyde, and combinations thereof.

In some embodiments, the method includes staining the biological sample, and optionally, where the staining includes use of eosin and/or hematoxylin. In some embodiments, the staining includes the use of a detectable label selected from the group consisting of a radioisotope, a fluorophore, a chemiluminescent compound, a bioluminescent compound, and a combination thereof.

In some embodiments, the method includes imaging the biological sample.

In some embodiments, the method includes before step (e), performing a step of extending a 3′ end of the one or more extension products of step (d) using the capture probe as a template, thereby generating one or more extended capture products, and/or performing a step of extending a 3′ end of the capture probes using the one or more extension products of step (d) as a template, thereby generating one or more extended capture probes.

In some embodiments, the one or more extended capture products are removed from the capture probe on the array. In some embodiments, the removing includes use of heat or KOH.

In some embodiments, the method includes generating a sequence library. In some embodiments, the determining in step (e) includes sequencing. In some embodiments, the sequencing includes high-throughput sequencing.

In some embodiments, the method includes a step of permeabilizing the biological sample. In some embodiments, the permeabilizing includes use of an organic solvent, a detergent, an enzyme, or a combination thereof. In some embodiments, the permeabilizing includes use of an endopeptidase, a protease sodium dodecyl sulfate, polyethylene glycol tert-octylphenyl ether, polysorbate 80, polysorbate 20, N-lauroyIsarcosine sodium salt solution, saponin, Triton X-100™, Tween-20™, or combinations thereof. In some embodiments, the endopeptidase is pepsin or proteinase K.

In some embodiments, the capture probe includes one or more functional domains, a unique molecular identifier, a cleavage domain, or combinations thereof.

In some embodiments, the biological sample is a tissue sample. In some embodiments, the tissue sample is a fixed tissue sample. In some embodiments, the fixed tissue sample is a formalin-fixed paraffin embedded tissue sample, a paraformaldehyde fixed tissue sample, a methanol fixed tissue sample, or an acetone fixed tissue sample. In some embodiments, the tissue sample is a fresh frozen tissue sample. In some embodiments, the biological sample is a tissue section. In some embodiments, the tissue section is a fixed tissue section. In some embodiments, the fixed tissue section is a formalin-fixed paraffin embedded tissue section, a paraformaldehyde fixed tissue section, a methanol fixed tissue section, or an acetone fixed tissue section.

In some embodiments, the plurality of primers hybridize to different sequences in the target nucleic acid. In some embodiments, two or more primers of the plurality of primers hybridize to different sequences in the target nucleic acid. In some embodiments, ten or more primers of the plurality of primers hybridize to different sequences in the target nucleic acid. In some embodiments, fifty or more primers of the plurality of primers hybridize to different sequences in the target nucleic acid. In some embodiments, one hundred or more primers of the plurality of primers hybridize to different sequences in the target nucleic acid.

In some embodiments, the extending in step (b) includes use of a reverse transcriptase. In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid. In some embodiments, the extending in step (b) includes the use of a reverse transcriptase and a helicase. In some embodiments, the method includes use of one or more single-stranded DNA binding proteins. In some embodiments, the one or more single-stranded DNA binding proteins includes one or more of: Tth RecA, E. coli RecA, T4 gp32 and ET-SSB. In some embodiments, the helicase has strand displacement activity. In some embodiments, the extending in step (b) includes the use of a superhelicase and a reverse transcriptase. In some embodiments, the superhelicase is selected from the group consisting of: Rep, PrcA, UvrB, RecBCD, and Tte-Uvrd. In some embodiments, the superhelicase has strand displacement activity.

In some embodiments, the method includes generating two or more extension products from a primer of the plurality of primers. In some embodiments, the one or more extension products comprise different sequence lengths.

In some embodiments, the functional sequence includes a primer binding sequence or a sequencing specific sequence.

Also provided herein are kits including: (a) an array including a plurality of capture probes, where a capture probe of the plurality of capture probes includes: (i) a spatial barcode; and (ii) a capture domain that binds a capture sequence, or a complement thereof; and (b) a plurality of primers, where a primer of the plurality of primers includes a sequence complementary to a target nucleic acid and a functional domain, wherein the functional domain comprises a primer binding sequence or a sequencing specific sequence.

In some embodiments, the capture probe includes one or more functional domains, a unique molecular identifier, a cleavage domain, or a combination thereof.

In some embodiments, the kit includes a reverse transcriptase with strand displacement activity. In some embodiments, the kit includes a polymerase.

In some embodiments, the capture domain includes a fixed sequence.

In some embodiments, the primer of the plurality of primers comprises a sequence complementary to a nucleic acid encoding an immune cell receptor.

In some embodiments, the kit includes a helicase. In some embodiments, the kit includes one or more single-stranded DNA binding proteins selected from the group comprising: Tth RecA, E. coli RecA, T4 gp32, and ET-SSB.

In some embodiments, the kit includes a superhelicase, where the superhelicase is selected from the group consisting of: Rep, PrcA, UvrB, RecBCD, and Tte-Uvrd.

Also provided herein are compositions including: a) a target nucleic acid encoding a B cell receptor or a T cell receptor; and b) one or more extension products hybridized to the target nucleic acid, where the one or more extension products each comprise in a 5′ to 3′ direction: i) a primer hybridized to the target nucleic acid in a region encoding a constant region of the B cell receptor or the T cell receptor, where the primer includes a functional domain; ii) a complementary sequence to the target nucleic acid preferably where the complementary sequence is complementary to a sequence encoding V and J sequences of the B cell receptor or T cell receptor; and iii) a capture sequence including a polynucleotide sequence.

In some embodiments, the composition includes a reverse transcriptase or a terminal deoxynucleotidyl transferase. In some embodiments, the composition includes a helicase. In some embodiments, the composition includes a single-stranded DNA binding protein selected from the group comprising: Tth RecA, E. coli RecA, T4 gp32, and ET-SSB. In some embodiments, the composition includes a superhelicase selected from the group consisting of: Rep, PrcA, UvrB, RecBCD, and Tte-Uvrd.

In some embodiments, the target nucleic acid is RNA. In some embodiments, the RNA is mRNA.

In some embodiments, the functional domain includes a primer binding site or a sequencing specific site.

In some embodiments, the complementary sequence is further complementary to a sequence of the target nucleic acid encoding a D sequence.

In some embodiments, the polynucleotide sequence comprised in the capture sequence is a heteropolynucleotide sequence. In some embodiments, the polynucleotide sequence includes a homopolynucleotide sequence, optionally where the homopolynucleotide sequence is 5′-CCC-3′.

In some embodiments, the composition includes two or more extension products hybridized to the target nucleic acid, wherein the two or more extension products each comprise a primer hybridized to a different region of the target nucleic acid.

Also provided herein are compositions including: one or more extension products where the one or more extension products comprise in a 5′ to 3′ direction including: i) a primer, where the primer includes a functional domain; ii) a complementary sequence, where the complementary sequence is complementary to a target nucleic acid that encodes V and J sequences of an immune cell receptor; and iii) a polynucleotide sequence, where the polynucleotide sequence is hybridized to a capture domain on an array, where the array includes a plurality of capture probes, where a capture probe of the plurality of capture probes includes a spatial barcode and the capture domain, and where the capture domain includes RNA. In some embodiments the RNA comprises three or more ribonucleotides.

In some embodiments, the capture probe includes a cleavage domain, one or more functional domains, a unique molecular identifier, or a combination thereof.

In some embodiments, the functional domain includes a primer binding sequence or a sequencing specific sequence.

In some embodiments, the complementary sequence of the target nucleic acid encodes a D sequence of an immune cell receptor.

In some embodiments, the polynucleotide sequence includes a homopolynucleotide sequence (e.g., 5′-CCC-3′). In some embodiments, the polynucleotide sequence includes a heteropolynucleotide sequence (e.g., 5′-CGC-3′).

Also provided herein are methods for processing a target nucleic acid in a biological sample, including: (a) contacting the biological sample with a primer including a nucleic acid sequence that hybridizes to a complementary sequence in the target nucleic acid and a functional domain; (b) hybridizing the primer to the target nucleic acid and extending the primer using the target nucleic acid as a template to generate an extension product; (c) incorporating a polynucleotide sequence including at least three nucleotides to the 3′ end of the extension product; and (d) hybridizing the polynucleotide sequence of the extension product to a capture domain on an array, where the array includes a plurality of capture probes, and where a capture probe of the plurality of capture probes includes a spatial barcode and the capture domain.

In some embodiments, the method includes (e) determining (i) the sequence of the spatial barcode, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

In any of the foregoing methods, the capture domain of the capture probe can comprises a sequence that is complemenatry to the polynucleotide sequence. In some embodiments, the sequence that is complemenatry to the polynucleotide sequence comprises RNA. In some preferred embodiments, the sequence that is complemenatry to the polynucleotide sequence comprises 5′-rGrGrG-3′or 5′-rGrCrG-3′.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram showing reverse transcription of a target nucleic acid with a primer to generate an extension product and the incorporation of a nontemplated polynucleotide sequence into the extension product which is capable of hybridizing to a capture domain of a capture probe.

FIG. 3 is a schematic diagram showing capture and extension on an array of the extension product (e.g., cDNA product) shown in FIG. 2 and extension of the capture probe and the captured extension product (e.g., cDNA product) followed by release of the extended capture product.

FIG. 4 is a schematic diagram showing reverse transcription of a target nucleic acid with a plurality of primers where the reverse transcription occurs with a reverse transcriptase with strand displacement activity or with a reverse transcriptase with a helicase or a superhelicase (top). In the embodiment shown, the primers hybridize to a region of the target nucleic acid that encodes for a constant region of an immune cell receptor and generate one or more extension products of varying lengths that include V(D)J sequences depending on where the primers hybridize to the target nucleic acid that encodes for a constant region of an immune cell receptor (bottom).

FIGS. 5A-B are schematic diagrams depicting exemplary sandwiching embodiments. FIG. 5A shows an exemplary sandwiching process where a first substrate including a biological sample and a second substrate are brought into proximity with one another and a liquid reagent drop is introduced on the second substrate in proximity to the capture probes and in between the biological sample. FIG. 5B shows a fully formed sandwich configuration creating a chamber formed from one or more spacers, the first substrate, and the second substrate including spatially barcoded capture probes.

### DETAILED DESCRIPTION

The present disclosure features methods of capturing analytes on a spatial array, where the spatial array includes a plurality of capture probes and a capture probe of the plurality of capture probes includes a spatial barcode and a capture domain. Sequences more than about 1 kilobase away from the 3′ end of nucleic acid analytes are generally not captured in nucleic acid sequencing libraries, however, some analytes, such as nucleic acid analytes encoding immune cell receptors (e.g., B cell receptor, T cell receptor) contain sequences of interest (e.g., VDJ sequences) more than 1 kilobase away from the 3′ end of the analyte. Since capture on a spatial array generally biases the 3′ end of nucleic acid analytes (e.g., mRNA), methods are needed to capture the 5′ end of the nucleic acid analyte, or a complement thereof (e.g., a proxy of the analyte).

Analytes, such as mRNA, can be reverse transcribed by hybridizing to the target mRNA a primer that includes a sequence complementary to the target nucleic acid and a functional domain (e.g., a sequence domain for use in sequencing, a primer binding domain, etc.) to generate an RNA/DNA (e.g., RNA/cDNA) duplex. A polynucleotide sequence can be incorporated at the end of the cDNA. For example, a reverse transcriptase or a terminal transferase can add a polynucleotide sequence in a template-independent manner (e.g., at least three non-templated nucleotides). In some embodiments, the polynucleotide sequence is a heteropolynucleotide sequence (e.g., 5′-CGC-3′). In some embodiments, the polynucleotide sequence is a homopolynucleotide sequence (e.g., 5′-CCC-3′). In some embodiments, the polynucleotide sequence added to the end of the cDNA is complementary to a capture domain (or a portion thereof) of a capture probe. In some embodiments, the polynucleotide sequence added to the end of the cDNA (e.g., a capture sequence) hybridizes to the capture domain of the capture probe. The target RNA can be removed (e.g., via digestion, denaturation, etc.) resulting in a single-stranded product that serves as a proxy of the target analyte which can be captured by a capture probe on a spatial array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3): 442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA). Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue sample. In some embodiments, a biological sample can be a tissue section. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

In some embodiments, a biological sample can be a fixed and/or stained biological sample (e.g., a fixed and/or stained tissue section). In some embodiments, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLOS One. ; 6(11): e27704 (2011); and Mathieson W. et al., Am J Clin Pathol. ; 146(1):25-40 (2016), each of which are hereby incorporated by reference in their entirety, for a description and evaluation of PAXgene for tissue fixation. Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

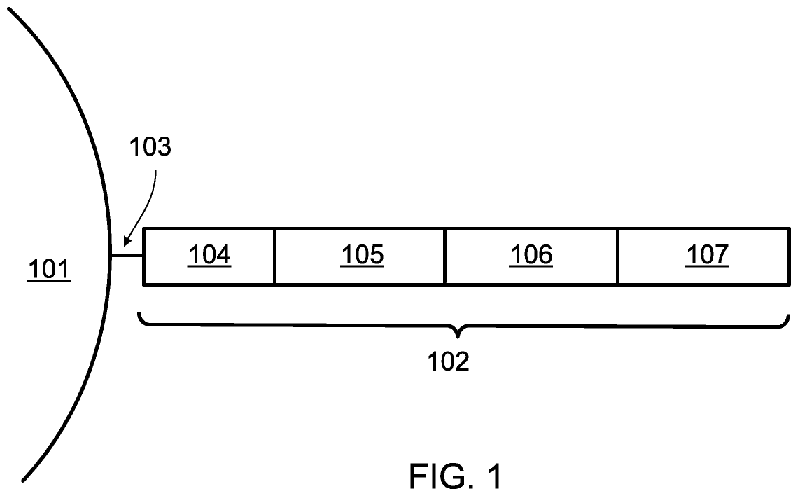

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to an analyte capture sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature (e.g., a bead, a well, a spot on an array). In some embodiments, the UMI sequence 106 of a capture probe attached to a given feature is different from the UMI sequence of a different capture probe attached to the given feature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., an extension product, a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to (also referred to as incorporating) a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

As used herein, “an extension product” refers to an analyte, such as RNA (e.g., mRNA), that has been reverse transcribed (e.g., with a reverse transcriptase) to generate cDNA. In some embodiments, the cDNA is hybridized to the RNA (e.g., RNA/cDNA duplex). In some embodiments, the cDNA is hybridized to the mRNA. In some embodiments, the extension product is further extended by adding a polynucleotide sequence (e.g., a heteropolynucleotide sequence, a homopolynucleotide sequence) to the 3′ end of the extension product (e.g., cDNA). For example, a reverse transcriptase or a terminal transferase can add at least three nucleotides (e.g., a polynucleotide sequence) to the 3′ end of the extension product in a template-independent manner. In some embodiments, the polynucleotide sequence added to the 3′ end of the extension product is complementary to a capture domain of a capture probe. In some embodiments, the polynucleotide sequence (e.g., a heteropolynucleotide sequence, a homopolynucleotide sequence) hybridizes to the capture domain of the capture probe. In some embodiments, the RNA (e.g., mRNA) is removed (e.g., by digestion) from the extension product. In such embodiments, the term extension product also refers to a single-stranded DNA (e.g., cDNA) product that includes a complement of the target nucleic acid (e.g., RNA, mRNA).

As used herein, “an extended capture product” refers to an extension product that has been captured on a spatial array and extended using the capture probe as a template. For example, an extension product, as described herein, after capture by a capture probe can be extended to include a capture sequence, or a complement thereof, that is capable of hybridizing to a capture domain of a capture probe. In some embodiments, when the extension product hybridizes to the capture domain of the capture probe, an end of the extension product (e.g., a 3′ end) can be extended to generate the extended capture product. In such examples, the extended capture product includes the domains (e.g., a UMI, a spatial barcode, one or more functional domains, or combination thereof etc.) present in the capture probe on the spatial array. In some embodiments, the extended capture product is released from the capture probe and collected for downstream applications, such as amplification and sequencing.

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe such as an extended capture product), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof), such as an extension product, can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### 5′ Capture of Target Nucleic Acids

The present disclosure features methods of capturing analytes on a spatial array, where the spatial array includes a plurality of capture probes and a capture probe of the plurality of capture probes includes a spatial barcode and a capture domain. In some embodiments, the analyte is a target nucleic acid. Since capture on a spatial array generally biases 3′ end sequences of nucleic acid analytes, methods are needed to determine the sequence of the 5′ end of nucleic acid analytes (e.g., by capturing the 5′ end of the nucleic acid analyte), or a complement thereof (e.g., a proxy of the analyte). For example, target nucleic acid analytes (e.g., RNA) can be reverse transcribed with a primer including a sequence complementary to the target nucleic acid and a functional domain, such as a primer binding site or a sequencing specific site, to generate an RNA/DNA (e.g., cDNA) duplex. An enzyme such as a reverse transcriptase or terminal transferase can add non-templated nucleotides to the 3′ end of the cDNA. For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA. In some embodiments, the polynucleotide sequence added to the end of the cDNA is complementary to a capture domain of a capture probe or a portion thereof. In some embodiments, the polynucleotide sequence added to the end of the cDNA (e.g., a capture sequence) hybridizes to the capture domain of the capture probe. The target nucleic acid can be removed (e.g., digested, denatured, etc.) resulting in a single-stranded DNA product. The single-stranded DNA product can include the functional domain at its 5′ end, a copy (e.g., complement) of the target analyte (e.g., cDNA), and the polynucleotide sequence at its 3′ end that is capable of binding (e.g., hybridizing) to a capture domain of a capture probe on the array.

In preferred embodiments of the methods of the disclosure, the extension reaction (e.g., reverse transcription) is performed in situ. For example, the biological sample containing the target nucleic acid is contacted with the primer or plurality of primers, such that the extension product(s) (e.g., cDNA) is generated in the biological sample.

Target nucleic acids can include a nucleic acid molecule with a nucleic acid sequence encoding at least a portion of a V-J sequence or a V(D)J sequence of an immune cell receptor (e.g., a T cell receptor or a B cell receptor). Target nucleic acids can include a nucleic acid molecule with a nucleic acid sequence encoding an antibody. In some embodiments, the target nucleic acid is RNA. In some embodiments, the RNA is mRNA. In some embodiments, the target nucleic acids are nucleic acids encoding immune cell receptors. In some embodiments, target nucleic acids encoding immune cell receptors identify clonotype populations from a biological sample. In some embodiments, target nucleic acids include a constant region, such as a sequence encoding a constant region of an immune cell receptor (e.g., an antibody). In some embodiments, target nucleic acids include a variable region, such as a sequence encoding a variable region of an immune cell receptor (e.g., antibody), for example, a sequence encoding CDR1, CDR2, and/or CDR3 of an immune cell receptor.

In some embodiments, the target nucleic acid encodes an immune cell receptor. In some embodiments, the immune cell receptor is a B cell receptor. In some embodiments, the B cell receptor includes an immunoglobulin kappa light chain. In some embodiments, the target nucleic acid includes a sequence encoding a CDR3 region of the immunoglobulin kappa light chain. In some embodiments, the target nucleic acid includes a sequence encoding one or both of CDR1 and CDR2 of the immunoglobulin kappa light chain. In some embodiments, the target nucleic acid includes a sequence encoding a full-length variable domain of the immunoglobulin kappa light chain.

In some embodiments, the B cell receptor includes an immunoglobulin lambda light chain. In some embodiments, the target nucleic acid includes a sequence encoding a CDR3 of the immunoglobulin lambda light chain. In some embodiments, the target nucleic acid includes a sequence encoding one or both of CDR1 and CDR2 of the immunoglobulin lambda light chain. In some embodiments, the target nucleic acid includes a sequence encoding a full-length variable domain of the immunoglobulin lambda light chain.

In some embodiments, the B cell receptor includes an immunoglobulin heavy chain. In some embodiments, the target nucleic acid includes a sequence encoding a CDR3 of the immunoglobulin heavy chain. In some embodiments, the target nucleic acid includes a sequence encoding one or both of CDR1 and CDR2 of the immunoglobulin heavy chain. In some embodiments, the target nucleic acid includes a sequence encoding a full-length variable domain of the immunoglobulin heavy chain.

In some embodiments, the immune cell receptor is a T cell receptor. In some embodiments, the T cell receptor includes a T cell receptor alpha chain. In some embodiments, the target nucleic acid includes a sequence encoding a CDR3 of the T cell receptor alpha chain. In some embodiments, the target nucleic acid includes a sequence encoding one or both of CDR1 and CDR2 of the T cell receptor alpha chain. In some embodiments, the target nucleic acid includes a sequence encoding a full-length variable domain of the T cell receptor alpha chain.

In some embodiments, the T cell receptor includes a T cell receptor beta chain. In some embodiments, the target nucleic acid includes a sequence encoding a CDR3 of the T cell receptor beta chain. In some embodiments, the target nucleic acid includes one or both of CDR1 and CDR2 of the T cell receptor beta chain. In some embodiments, the target nucleic acid further includes a full-length variable domain of the T cell receptor beta chain.

While useful for analysis of immune cell receptor analytes such as nucleic acids encoding immune cell receptors, the disclosed methods are not limited thereto. Thus, the disclosed methods can be useful for analysis of nucleotide sequence at and/or proximal to the 5′ end of any target nucleic acid, such as, genomic DNA, IncRNA, and the like.

In some embodiments, the methods included herein include determining the complementarity determining regions (CDRs) of a T cell receptor or a fragment thereof, and/or a B cell receptor or a fragment thereof (e.g., an antibody or fragment thereof).

Thus, provided herein are methods for determining a location of a target nucleic acid in a biological sample, the method including: (a) contacting the biological sample with a primer including a nucleic acid sequence that hybridizes to a complementary sequence in the target nucleic acid and a functional domain; (b) hybridizing the primer to the target nucleic acid and extending the primer using the target nucleic acid as a template to generate an extension product; (c) adding a non-templated sequence (e.g., a polynucleotide sequence including at least three nucleotides) to the 3′ end of the extension product; (d) hybridizing the polynucleotide sequence of the extension product to a capture domain on an array, where the array includes a plurality of capture probes, and where a capture probe of the plurality of capture probes includes a spatial barcode and the capture domain; and (e) determining (i) the sequence of the spatial barcode, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

The methods described herein can also use a plurality of primers. For example, the plurality of primers can hybridize to a target nucleic acid at different locations in the target nucleic acid and subsequently be extended. Extending the plurality of primers generates one or more extension products that include a polynucleotide sequence complementary to a capture domain of a capture probe. For example, the methods provided herein can include providing a plurality of primers wherein each primer includes a sequence that hybridizes to a complementary sequence in the target nucleic acid and a functional domain and (i) hybridizing the plurality of primers to the target nucleic acid and extending one or more primers from the plurality of primers using the target nucleic acid as a template to generate one or more extension products; (ii) incorporating a non-templated sequence (e.g., a polynucleotide sequence including at least three nucleotides) to the 3′ end of the one or more extension products; (iii) hybridizing the non-templated sequence of the one or more extension products to a capture domain on an array, where the array includes a plurality of capture probes, and where a capture probe of the plurality of capture probes comprises a spatial barcode and the capture domain; and (iv) determining (1) the sequence of the spatial barcode, or a complement thereof, and (2) all or a portion of the one or more extension products corresponding to the sequence of the target nucleic acid, or a complement thereof, and using the determined sequences of (1) and (2) to determine the location of the target nucleic acid in the biological sample.

Also provided herein are methods for determining locations of target nucleic acids in a biological sample, the method including: (a) contacting the biological sample with a plurality of primers, where the plurality of primers comprise nucleic acid sequences that hybridize to complementary sequences in the target nucleic acids and a functional domain; (b) hybridizing the plurality of primers to the target nucleic acids and extending one or more of the plurality of primers using the target nucleic acids as a template to generate one or more extension products; (c) adding a non-templated sequence (e.g., a polynucleotide sequence comprising at least three nucleotides) to the 3′ end of the one or more extension products; (d) hybridizing the non-templated sequence (e.g., polynucleotide sequence) of the one or more extension products of (c) to a plurality of capture domains on an array, wherein the array comprises a plurality of capture probes, and wherein the plurality of capture probes comprise a spatial barcode and a capture domain; and (e) determining (i) the sequences of the spatial barcodes, or a complement thereof, and (ii) all or a portion of the sequence of the target nucleic acids, or a complement thereof, and using the determined sequences of (i) and (ii) to determine the locations of the target nucleic acids in the biological sample.

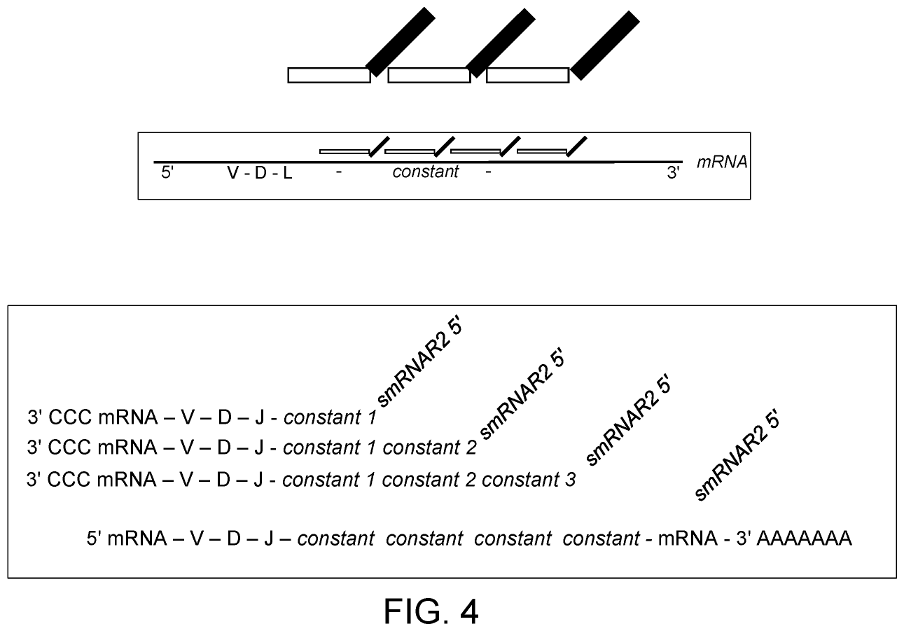

In embodiments where a plurality of primers are used, the plurality of primers can hybridize to different sequences in the target nucleic acid. For example, the plurality of primers can hybridize to adjacent sequences (e.g., “tiling”) on the target nucleic acid. Adjacent sequences can be, but are not necessarily, contiguous. In some embodiments, adjacent sequences can be within 1-10 nucleotides of each other. In some embodiments, the plurality of primers hybridize to non-adjacent sequences on the target nucleic acid. In some embodiments, two or more primers of the plurality of primers hybridize to two or more different sequences in the target nucleic acid. In some embodiments, ten or more primers of the plurality of primers hybridize to ten or more different sequences in the target nucleic acid. In some embodiments, fifty or more primers of the plurality of primers hybridize to fifty or more different sequences in the target nucleic acid. In some embodiments, one hundred or more primers of the plurality of primers hybridize to one hundred or more different sequences in the target nucleic acid. In some embodiments, a plurality of extension products can be generated by extension of the plurality of primers. The resulting extension products can have differing lengths depending on the exact location in the target nucleic acid from which they were primed. Thus, in some embodiments, extension products (e.g., cDNAs) of different lengths and/or sequences can be generated from the same target nucleic acid (e.g., mRNA). See, e.g., FIG. 4.

In embodiments where the target nucleic acid includes a sequence that encodes an immune cell receptor, the plurality of primers preferably hybridize to a region of the target nucleic acid that encodes a constant region of the immune cell receptor.

In some embodiments, the extension product(s) that hybridize to the capture domains of the plurality of capture probes can migrate (e.g., diffuse) towards the capture probes through passive migration such as gravity. In some embodiments, the extension product(s) that hybridize to the capture domains of the plurality of capture probes can migrate toward the capture probes through active migration. In some embodiments, the active migration includes electrophoresis.

In some embodiments, the array containing the plurality of capture probes includes one or more features (e.g., any of the features described herein). In some embodiments, the one or more features includes a bead.

Also provided herein are methods for processing a target nucleic acid in a biological sample, including: (a) contacting the biological sample with a primer including a nucleic acid sequence that hybridizes to a complementary sequence in the target nucleic acid and a functional domain; (b) hybridizing the primer to the target nucleic acid and extending the primer using the target nucleic acid as a template to generate an extension product; (c) adding a polynucleotide sequence including at least three nucleotides to the 3′ end of the extension product; and (d) hybridizing the polynucleotide sequence of the extension product to a capture domain on an array, where the array includes a plurality of capture probes, and where a capture probe of the plurality of capture probes includes a spatial barcode and the capture domain.

In some embodiments, the method includes (e) determining (i) the sequence of the spatial barcode, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

In some embodiments of the methods of the disclosure, the capture domain of the capture probe comprises a sequence that is complemenatry to the polynucleotide sequence. In some embodiments, the sequence of the capture domain that is complemenatry to the polynucleotide sequence comprises RNA. In some preferred embodiments, the sequence of the capture domain that is complemenatry to the polynucleotide sequence comprises 5′-rGrGrG-3′ or 5′-rGrCrG-3′.

As described herein, a capture probe refers to any molecule capable of capturing (directly or indirectly) and/or labelling an analyte (e.g., an analyte of interest, such as a nucleic acid) in a biological sample. In some embodiments, the capture probe is a nucleic acid. In some embodiments, the capture probe is DNA. In some embodiments, the capture probe includes a barcode (e.g., a spatial barcode) and/or a unique molecular identifier (UMI)) and a capture domain. In some embodiments, a capture probe can include a cleavage domain and/or one or more functional domains (e.g., a primer-binding site or a sequencing specific site, such as for next-generation sequencing (NGS)).

In some embodiments, the capture domain includes a fixed sequence. As used herein, a “fixed sequence” is a non-random sequence. A “fixed sequence” can be complementary to the non-templated sequence that is incorporated into the extension product(s) described herein. For example, the capture domain (e.g., the fixed sequence therein) or a portion thereof can be complementary to the non-templated polynucleotide sequence added to the extension product(s). In some embodiments, the plurality of capture probes on an array include the same fixed sequence. The methods described herein can use different capture sequences which are incorporated (e.g., a complement thereof) into the extension product(s). For example, it is only necessary for the polynucleotide sequence added to the extension products and the capture domain to be substantially complementary to each other, such that the capture domain including a fixed sequence can hybridize to and/or capture the extension product(s).

In some embodiments, the primer includes a sequence that is complementary to a sequence in a target nucleic acid. The sequence complementary to the target nucleic acid can be a gene specific sequence, which, for example, may allow for selective capture of a desired target nucleic acid. In some embodiments, the primer includes a homopolymer sequence and a functional domain. In some embodiments, the homopolymer sequence is a poly(T) sequence. In some embodiments, the primer includes a random sequence and a functional domain. In some embodiments, the random sequence comprises 5, 6, 7, 8, 9, 10, 11, 12, 13, 14, 15, 16, 17, 18, 19, 20, or more random nucleotides. In some embodiments, the random sequence is a random hexamer. In some embodiments, the random sequence is a random decamer. In some embodiments, the functional domain is a primer binding site. In some embodiments, the functional domain is a sequencing specific site.

In some embodiments, generation of the extension product in step (b) includes the use of a reverse transcriptase. In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid.

In some embodiments, the extending in step (b) includes the use of a reverse transcriptase and a helicase. As defined herein, “helicases” are enzymes that catalyze the reaction of separating or unwinding the helical structure of nucleic acid complexes, e.g. double-stranded DNA, double-stranded RNA, or DNA:RNA complexes, into single stranded components. Helicases generally are known to use a nucleoside triphosphate (NTP) (e.g., ATP) hydrolysis as a source of energy. In some embodiments, the method includes use of one or more single-stranded DNA binding proteins. In some embodiments, the one or more single-stranded DNA binding proteins comprises one or more of: Tth RecA, E. coli RecA, T4 gp32, and ET-SSB. As defined herein, “single-stranded DNA binding proteins” or “SSBs” are proteins that bind to single-stranded DNA. SSBs, or functional equivalents, are found in a variety of organisms, including eukaryotes and bacteria. Single-stranded DNA is produced, for example, during aspects of DNA metabolism, DNA replication, DNA recombination, and DNA repair. As well as stabilizing single-stranded DNA, SSB proteins bind to and modulate the function of numerous proteins involved in the aforementioned processes. In addition, SSB proteins can destabilize ends of double-stranded nucleic acid (e.g., dsDNA). In some embodiments, the helicase has strand displacement activity. For example, the helicases and single-stranded DNA binding proteins described herein can unwind DNA:RNA complexes that allow a reverse transcriptase to reverse transcribe the target nucleic acid. In some embodiments, a helicase can unwind a first target nucleic acid: extension product complex (e.g., mRNA:cDNA) that is downstream of a second target nucleic acid: extension product complex, such that the target nucleic acid from the first complex (with or without the help of the SSBs) becomes available as template for further extension of the extension product in the second complex.

In some embodiments, helicases, including superhelicases, are also used in conjunction with SSB proteins during nucleic acid amplification. In some embodiments, the extending in step (b) comprises the use of a superhelicase and a reverse transcriptase. In some embodiments, the superhelicase is selected from the group consisting of: Rep, PrcA, UvrB, RecBCD, and Tte-Uvrd. As defined herein a “superhelicase” is a mutant and/or a derivative of a helicase. Superhelicases can have increased processivity compared to helicases due to one or more derivations that can include mutated gene or substituted polypeptide sequences and/or cross-linked protein domains. Additionally, superhelicases can unwind double-stranded nucleic acid complexes without single-stranded DNA binding protein(s). In some embodiments, the helicase used in the disclosed methods is a superhelicase. In some embodiments, superhelicases have increased processivity relative to helicases. In some embodiments, the superhelicase has strand displacement activity. For example, superhelicases can unwind double-stranded nucleic acid complexes longer than 150 base pairs.

In some embodiments, the method includes generating two or more extension products from the primer of the plurality of primers. For example, a single primer can facilitate reverse transcription for more than a single extension reaction resulting in 2, 3, 4, 5, 6, 7, 8, 9, 10, 20, 30, 40, 50, 100, 200, 300, 400, 500, 600, 700, 800, 900, 1000, or more extension products produced from a single primer. In some embodiments, the two or more extension products generated from the same primer comprise the same sequence and/or length.

In some embodiments, the plurality of primers comprises multiple unique primers, wherein each unique primer comprises a unique sequence. The plurality of primers may comprise multiple copies (e.g., 2, 10, 20, 50, 100, 1000, 10000 or more) of each unique primer. In some embodiments, each unique primer in the plurality of primers hybridizes to a distinct sequence within the target nucleic acid. Thus, in some embodiments, the one or more extension products comprise different sequences and/or lengths. For example, when a plurality of primers are hybridized to a target nucleic acid and extended, the one or more extension products can have varying sequences and/or lengths.

In some embodiments, the extending in step (b) includes the use of a reverse transcriptase (e.g., any suitable reverse transcriptase known in the art). In some embodiments, adding the polynucleotide sequence to the 3′ end of the extension product in step (c) includes the use of the reverse transcriptase. In some embodiments, adding the polynucleotide sequence to the 3′ end of the extension product in step (c) includes the use of a terminal transferase. In some embodiments, the terminal transferase is terminal deoxynucleotidyl transferase. In some embodiments, the reverse transcriptase or the terminal transferase adds at least three nucleotides to the extension product. In some embodiments, the reverse transcriptase or the terminal transferase adds 4, 5, 6, 7, 8, 9, 10, or more nucleotides (e.g., a polynucleotide sequence) to the 3′ end of the extension product(s) in step (c). In some embodiments, the polynucleotide sequence added to the 3′ end of the extension product(s) is 5′-CCC-3′. In some embodiments, the polynucleotide sequence added to the 3′ end of the extension product(s) is 5′-CGC-3′.

In some embodiments, the method includes removing the target nucleic acid, or any other nucleic acid hybridized to the extension product(s) (e.g., extended cDNA product) before the sequence added to the extension product (e.g., a homopolynucleotide sequence, a heteropolynucleotide sequence) hybridizes to the capture domain of the capture probe. In some embodiments, the removing includes the use of an RNase. In some embodiments, the RNase is RNase A. In some embodiments, the RNase is RNase P. In some embodiments, the RNase is RNase T1. In some embodiments, the RNase is RNase H. In some embodiments, the removing includes use of heat.

In some embodiments, the extension product(s) (e.g., the single-stranded extension product(s)) hybridizes to the capture domain of the capture probe on the substrate. In some embodiments, the 3′ end of the capture probe is extended using the extension product as a template. In some examples, the 3′ end of the extension product (e.g., single-stranded cDNA product) is extended using the capture probe as a template, thereby generating an extended capture product. In some embodiments, both the capture probe, and the extension products hybridized thereto, are extended from their 3′ ends. In some embodiments, the extending includes the use of a polymerase. Any suitable polymerase can be used (e.g., Kapa Hifi). In some examples, the 3′ end of the capture probe is extended using the extension product as a template and the 3′ end of the extension product is simultaneously extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

In some embodiments, the released, extended captured products can be prepared for downstream applications, such as generation of a sequencing library and next-generation sequencing. Generating sequencing libraries are known in the art. For example, the extended captured products can be purified and collected for downstream amplification steps. The extended amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the target nucleic acid sequence.

Alternatively or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flowcell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for performing the methods as disclosed herein to the biological sample.

In some embodiments, the method includes staining the biological sample. In some embodiments, the staining includes the use of hematoxylin and/or eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, the method includes a step of permeabilizing the biological sample (e.g., a tissue section). For example, the biological sample can be permeabilized to facilitate transfer of the extended products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

In some embodiments, the biological sample is a tissue sample. In some embodiments, the tissue sample is a fixed tissue sample. For example, fixing the biological sample can include the use of a fixative including: ethanol, methanol, acetone, formaldehyde, paraformaldehyde-Triton, glutaraldehyde, and combinations thereof. In some embodiments, the fixed tissue sample is a formalin-fixed paraffin embedded tissue sample, a paraformaldehyde fixed tissue sample, a methanol fixed tissue sample, or an acetone fixed tissue sample. In some embodiments, the tissue sample is a fresh frozen tissue sample. In some embodiments, the biological sample is a tissue section. In some embodiments, the biological sample is a fixed tissue section (e.g., a fixed tissue section prepared by any of the methods described herein).

In some embodiments, the method includes generating a sequencing library. In some embodiments, the determining in step (e) includes sequencing. Methods and systems for sequencing are known in the art and are described herein. In some embodiments, the sequencing is high-throughput sequencing. In some embodiments, the sequencing is long read sequencing.

### Sandwich Configurations

Some steps of the methods described herein can be performed in a biological sample (e.g., in situ) prior to contacting (or bringing into proximity) the biological sample with the array including a plurality of capture probes. In some embodiments, the biological sample is disposed or placed on the array including the plurality of capture probes prior to step (a). In some embodiments, the biological sample is disposed or placed on the array including the plurality of capture probes prior to step (d). In some embodiments, the biological sample is not disposed or placed on the array. For example, the biological sample can be placed on a substrate (e.g., a glass slide) that does not include a spatial array. In some embodiments, the substrate including the biological sample can be aligned with the array (e.g., “sandwiched”) such that at least a portion of the biological sample is aligned with at least a portion of the array. In embodiments where the biological sample is disposed or placed on a substrate, steps (a)-(c) can be performed prior to aligning the substrate with the array as described herein. In embodiments where the biological sample is disposed or placed on a substrate, steps (a)-(c) can be performed after aligning the substrate with the array as described herein.

In some embodiments, one or more extension products generated from the biological sample are released from the biological sample and migrate to a substrate comprising an array of capture probes where the extension product(s) hybridize to the capture probes of the array. In some embodiments, the release and migration of the extension product(s) to the substrate comprising the array of capture probes occurs in a manner that preserves the original spatial context of the extension product(s) in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., U.S. Patent Application Pub. No. 20210189475, WO 2021/252747, and WO 2022/061152A. In some embodiments, the sandwiching process may be facilitated by a device, sample holder, sample handling apparatus, or system described in, e.g., U.S. Patent Application Pub. No. 20210189475, WO 2021/252747, or WO 2022/061152A.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the extension product(s) from the biological sample.

The sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to a to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

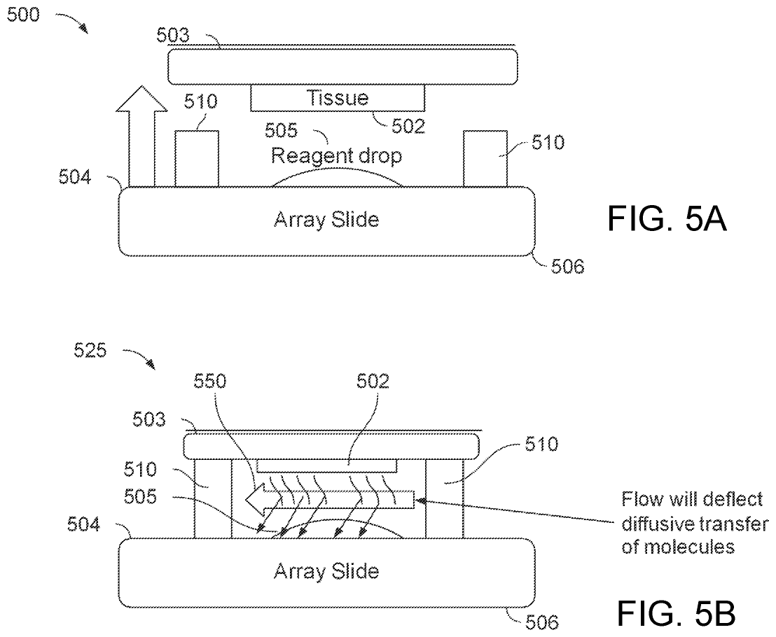

FIG. 5A shows an exemplary sandwiching process 500 where a first substrate (e.g., slide 503), including a biological sample 502 (e.g., a tissue section), and a second substrate (e.g., slide 504 including spatially barcoded capture probes 506) are brought into proximity with one another. As shown in FIG. 5A a liquid reagent drop (e.g., permeabilization solution 505) is introduced on the second substrate in proximity to the capture probes 506 and in between the biological sample 502 and the second substrate (e.g., slide 504 including spatially barcoded capture probes 506). The permeabilization solution 505 can release extension product(s) that can be captured (e.g., hybridized) by the capture probes 506 of the array. As further shown, one or more spacers 510 can be positioned between the first substrate (e.g., slide 503) and the second substrate (e.g., slide 504 including spatially barcoded capture probes 506). The one or more spacers 510 may be configured to maintain a separation distance between the first substrate and the second substrate. While the one or more spacers 510 is shown as disposed on the second substrate, the spacer may additionally or alternatively be disposed on the first substrate.

FIG. 5B shows a fully formed sandwich configuration creating a chamber 550 formed from the one or more spacers 510, the first substrate (e.g., the slide 503), and the second substrate (e.g., the slide 504 including spatially barcoded capture probes 506) in accordance with some example implementations. In FIG. 5B, the liquid reagent (e.g., the permeabilization solution 505) fills the volume of the chamber 550 and can create a permeabilization buffer that allows extension product(s) to diffuse from the biological sample 502 toward the capture probes 506 of the second substrate (e.g., slide 504). In some aspects, flow of the permeabilization buffer may deflect extension product(s) from the biological sample 502 and can affect diffusive transfer of extension product(s) for spatial analysis. A partially or fully sealed chamber 550 resulting from the one or more spacers 510, the first substrate, and the second substrate can reduce or prevent flow from undesirable convective movement of transcripts and/or molecules over the diffusive transfer from the biological sample 502 to the capture probes 506.

### Compositions

Also provided herein are compositions including a) a target nucleic acid encoding an immune cell receptor (e.g., B cell receptor or a T cell receptor); and b) one or more extension products hybridized to the target nucleic acid, wherein the one or more extension products each comprises in a 5′ to 3′ direction: i) a primer hybridized to the target nucleic acid in a region encoding a constant region of the immune cell receptor (e.g., B cell receptor or a T cell receptor), where the primer comprises a functional domain; ii) a sequence complementary to a region of the target nucleic acid that encodes the variable region of the immune cell receptor (e.g., V and/or J sequences); and iii) a capture sequence comprising a polynucleotide sequence (e.g., a non-templated sequence that is at least three nucleotides in length).

In some embodiments, the target nucleic acid is RNA. In some embodiments, the RNA is mRNA. In some embodiments, the mRNA includes a sequence encoding an antibody or a fragment thereof. In some embodiments, the mRNA includes a sequence encoding a T cell receptor or a fragment thereof. In some embodiments, the mRNA includes a sequence encoding a B cell receptor.

In some embodiments, the functional domain includes a primer binding site. In some embodiments, the functional domain includes a sequencing specific site.

In some embodiments, the composition includes a reverse transcriptase. In some embodiments, the composition includes a helicase. In some embodiments, the composition includes one or more single-stranded DNA binding proteins selected from the group comprising: Tth RecA, E. coli RecA, T4 gp32, and ET-SSB. In some embodiments, the composition includes a superhelicase selected from the group consisting of: Rep, PrcA, UvrB, RecBCD, and Tte-Uvrd.

In some embodiments, the polynucleotide sequence includes a homopolynucleotide sequence (e.g., 5′-CCC-3′). In some embodiments, the polynucleotide sequence includes a heteropolynucleotide sequence (e.g., 5′-CGC-3′).

In some embodiments, the extension products further include a sequence complementary to a region of the target nucleic acid that encodes a D sequence of an immune cell receptor.

Also provided herein are compositions including one or more extension products where the one or more extension products comprise in a 5′ to 3′ direction including: i) a primer, wherein the primer includes a functional domain; ii) a sequence complementary to a region of a target nucleic acid that encodes V and/or J sequences of an immune cell receptor; and iii) a polynucleotide sequence, where the polynucleotide sequence is hybridized to a capture domain on an array, wherein the array comprises a plurality of capture probes, and where a capture probe of the plurality of capture probes comprises a spatial barcode and the capture domain. In some embodiments, the capture domain comprises RNA. In some embodiments, the RNA comprises three or more ribonucleotides.

In some embodiments, the polynucleotide sequence is a heteropolynucleotide sequence. In some embodiments, the polynucleotide sequence is a homopolynucleotide sequence.

In some embodiments, the capture probe includes a cleavage domain, one or more functional domains, a unique molecular identifier, or a combination thereof.

In some embodiments, the functional domain comprises a primer binding sequence or a sequencing specific sequence.

In some embodiments, the one or more extension products further include a sequence complementary to a region of the target nucleic acid that encodes a D sequence of an immune cell receptor.

In some embodiments, the target nucleic acid is RNA. In some embodiments, the RNA is mRNA.

In some embodiments, the primer includes a homopolymer sequence and a functional domain. In some embodiments, the homopolymer sequence is a poly(T) sequence. In some embodiments, the primer includes a random sequence and a functional domain. In some embodiments, the random sequence includes 5, 6, 7, 8, 9, 10, 11, 12, 13, 14, 15, 16, 17, 18, 19, 20, or more random nucleotides. In some embodiments, the random sequence is a random hexamer. In some embodiments, the random sequence is a random decamer. In some embodiments, the functional domain includes a primer binding site. In some embodiments, the functional domain includes a sequencing specific site.

### Kits

Also provided here are kits including (a) an array including a plurality of capture probes, where a capture probe of the plurality of capture probes includes: (i) a spatial barcode; and (ii) a capture domain that binds a capture sequence, or a complement thereof; (b) a plurality of primers wherein a primer of the plurality of primers comprises a sequence complementary to a target nucleic acid and a functional domain, where the functional domain comprises a primer binding sequence or a sequencing specific sequence. In some embodiments, the primer of the plurality of primers comprises a sequence complementary to a nucleic acid encoding an immune cell receptor.

In some embodiments, the capture probe includes one or more functional domains, a unique molecular identifier, a cleavage domain, or a combination thereof.

In some embodiments, the kit includes a reverse transcriptase. In some embodiments, the reverse transcriptase has strand displacement activity. In some embodiments, the kit includes a terminal deoxynucleotidyl transferase. In some embodiments, the kit includes a polymerase.

In some embodiments, the capture domain comprises a fixed sequence as described herein.

In some embodiments, the kit includes a helicase. In some embodiments, the kit includes one or more single-stranded DNA binding proteins such as one or more of: Tth RecA, E. coli RecA, T4 gp32 and ET-SSB. In some embodiments, the kit further includes a superhelicase. The superhelicase may be selected from the group consisting of: Rep, PrcA, UvrB, RecBCD, and Tte-Uvrd.

### Example 1.5′ Capture of Target Nucleic Acids

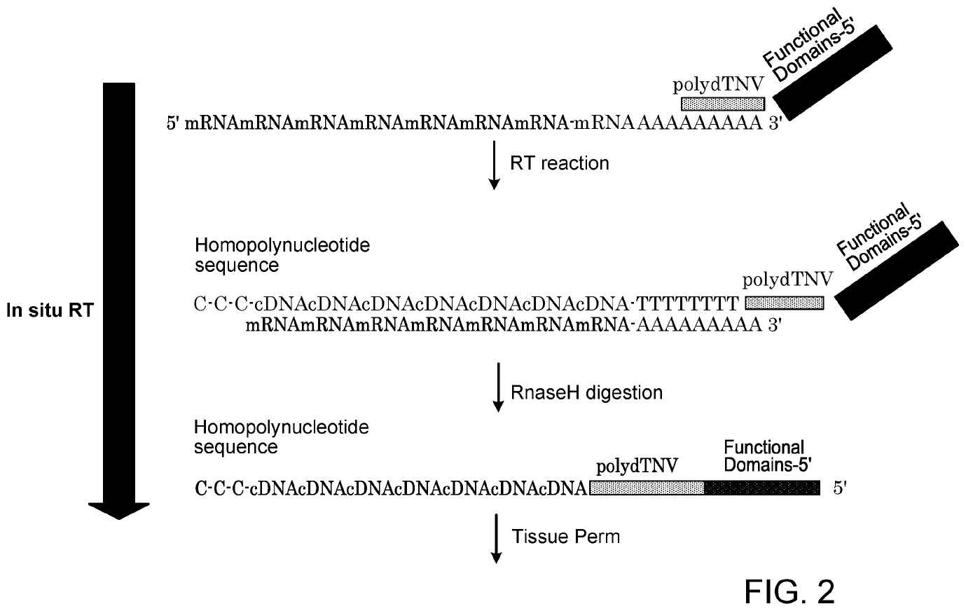

FIG. 2 is a schematic showing generation of a cDNA by in situ reverse transcription of a target nucleic acid (e.g., mRNA) from a primer including a sequence complementary to the target nucleic acid and a functional domain. More specifically, a biological sample containing a target nucleic acid is contacted with a primer that includes a sequence complementary to the target nucleic acid (e.g., poly(dT) sequence, a poly(dTNV) sequence, a random sequence, a sequence encoding a constant region of a B cell receptor (e.g., an antibody), or a T cell receptor) and a functional domain. In some examples, the functional domain is a primer binding site. In some examples, the functional domain is a sequencing specific site (e.g., Read2 site). The target nucleic acid is reverse transcribed into cDNA and a polynucleotide sequence is added to the 3′ end of the cDNA. FIG. 2 shows a homopolynucleotide sequence comprising cytosines, however, it is appreciated that other polynucleotide sequences can be added to the 3′ end of the cDNA, including a heteropolynucleotide sequence, such as CGC. In some examples, the polynucleotide sequence is added by the reverse transcriptase. In some examples, the polynucleotide sequence is added by a terminal transferase (e.g., terminal deoxynucleotidyl transferase).

After reverse transcription and incorporation of the polynucleotide sequence at the 3′ end of the cDNA, an RNase (e.g., RNase H) is contacted with the biological sample (e.g., a tissue section). The RNase degrades the RNA strand of the RNA/cDNA duplex, leaving a single-stranded cDNA product (e.g., an extension product) that includes the primer at its 5′ end and the added polynucleotide sequence which is substantially complementary to and capable of hybridizing to a sequence in a capture domain of a capture probe.

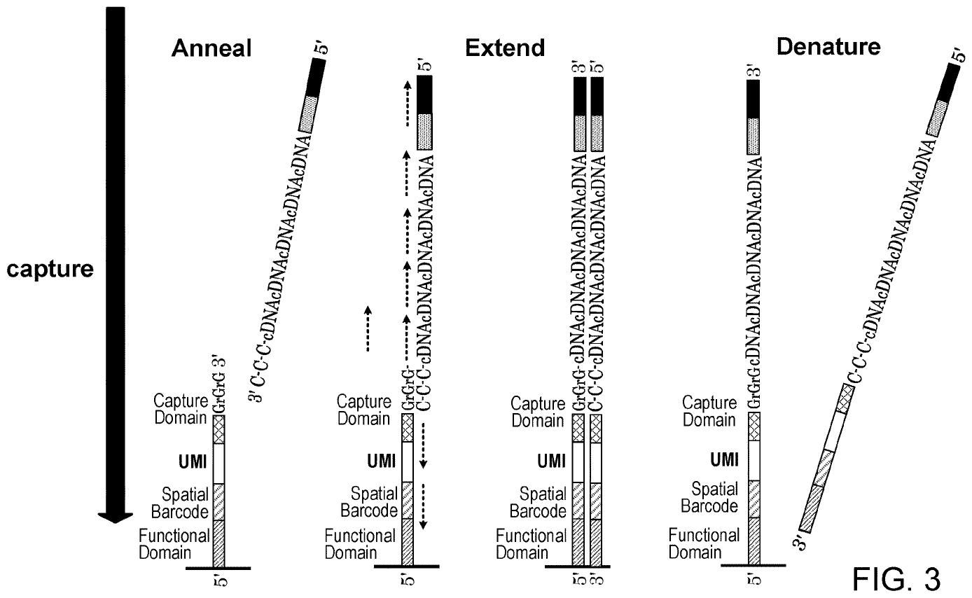

FIG. 3 is a schematic showing capture of the extension product (e.g., the single-stranded cDNA product shown in FIG. 2) by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hy bridizes to the added polynucleotide sequence (which is substantially complementary to and capable of hybridizing to a capture domain of a capture probe) within the extension product (e.g., single-stranded cDNA product) from FIG. 2. In some examples, the 3′ end of the capture probe is extended using the extension product as a template, thereby generating an extended capture probe. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

FIG. 4 is a schematic diagram showing an embodiment of FIG. 2 where reverse transcription of a target nucleic acid is performed with a plurality of primers. In some examples, reverse transcription is performed using a reverse transcriptase with strand displacement activity. In some examples, reverse transcription is performed with a reverse transcriptase and a helicase. In some examples, reverse transcription is performed with a reverse transcriptase and a superhelicase. In some examples, reverse transcription is performed with one or more single-stranded DNA binding proteins. When a plurality of primers are used to template reverse transcription as shown in FIG. 4, the resulting extension products can be of different lengths depending on where the primer hybridized to the target nucleic acid (bottom of FIG. 4). In some examples, a primer of the plurality of primers can facilitate more than one reverse transcription reaction, thus resulting in two or more extension products generated from the same primer.

The released, extended captured products can be prepared for downstream applications, such as generation of a sequencing library and next-generation sequencing.

What is claimed is:

1. A method for determining a location of a target nucleic acid in a biological sample, the method comprising:
(a) contacting the biological sample with a primer comprising a nucleic acid sequence that hy bridizes to a complementary sequence in the target nucleic acid and a functional domain;
(b) hybridizing the primer to the target nucleic acid and extending the primer using the target nucleic acid as a template to generate an extension product;
(c) incorporating a polynucleotide sequence comprising at least three nucleotides to the 3′ end of the extension product;
(d) hybridizing the polynucleotide sequence of the extension product to a capture domain on an array, wherein the array comprises a plurality of capture probes, and wherein a capture probe of the plurality of capture probes comprises a spatial barcode and the capture domain; and
(e) determining (i) the sequence of the spatial barcode, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

2. The method of claim 1, wherein the biological sample is disposed on the array comprising the plurality of capture probes.

3. The method of claim 1, wherein the biological sample is disposed on a substrate, preferably wherein the substrate does not comprise the array comprising the plurality of capture probes.

4. The method of claim 3, wherein the method further comprises aligning the substrate with the array, such that a least a portion of the biological sample is aligned with at least a portion of the array.

5. The method of claim 3 or 4, wherein the hybridizing in step (d) is facilitated by passive migration of the extension product to the array.

6. The method of claim 3 or 4, wherein the hybridizing in step (d) is facilitated by active migration of the extension product to the array, and optionally, wherein the active migration comprises electrophoresis.

7. The method of any one of claims 1-6, wherein the polynucleotide sequence in step (c) comprises a homopolynucleotide sequence, optionally 5′-CCC-3′.

8. The method of any one of claims 1-6, wherein the polynucleotide sequence in step (c) comprises a heteropolynucleotide sequence, optionally 5′-CGC-3′.

9. The method of any one of claims 1-8, wherein the capture domain comprises a fixed sequence.

10. The method of any one of claims 1-9, wherein the primer comprises a random sequence.

11. The method of claim 10, wherein the random sequence comprises a random hexamer.

12. The method of claim 10, wherein the random sequence comprises a random decamer.

13. The method of any one of claims 1-9, wherein the primer comprises a homopolymer sequence.

14. The method of claim 13, wherein the homopolymer sequence is a poly(T) sequence.

15. The method of any one of claims 1-9, wherein the primer comprises a sequence complementary to a nucleic acid sequence encoding a constant region of an immune cell receptor.

16. The method of any one of claims 1-9, wherein the primer comprises a sequence complementary to a nucleic acid sequence encoding a constant region of a B cell receptor.

17. The method of any one of claims 1-9, wherein the primer comprises a sequence complementary to a nucleic acid sequence encoding a constant region of a T cell receptor.

18. The method of any one of claims 1-17, wherein the array comprises one or more features.

19. The method of any one of claims 1-18, wherein the functional domain comprises a primer binding site or a sequencing specific site.

20. The method of any one of claims 1-19, wherein the target nucleic acid is an RNA.

21. The method of claim 20, wherein the RNA is mRNA.

22. The method of claim 21, wherein the mRNA comprises a sequence encoding a T cell receptor.

23. The method of claim 21, wherein the mRNA comprises a sequence encoding a B cell receptor.

24. The method of any one of claims 1-23, wherein incorporating the polynucleotide sequence to the 3′ end of the extension product in step (c) comprises use of a terminal deoxynucleotidyl transferase.

25. The method of any one of claims 1-23, wherein incorporating the polynucleotide sequence to the 3′ end of the extension product in step (c) comprises use of a reverse transcriptase.

26. The method of any one of claims 1-25, wherein the method further comprises removing the target nucleic acid, or any other nucleic acid hybridized to the extension product, before the polynucleotide sequence hybridizes to the capture domain of the capture probe on the array in step (d).

27. The method of claim 26, wherein the removing comprises the use of an RNase.

28. The method of claim 27, wherein the RNase is RNaseH.

29. The method of claim 26, wherein the removing comprises the use of heat.

30. The method of any one of claims 1-29, wherein the method further comprises fixing the biological sample.

31. The method of claim 30, wherein fixing the biological sample comprises the use of a fixative selected from the group consisting of: ethanol, methanol, acetone, formaldehyde, paraformaldehyde-Triton, glutaraldehyde, and combinations thereof.

32. The method of any one of claims 1-31, wherein the method further comprises staining the biological sample.

33. The method of claim 32, wherein the staining comprises use of eosin and/or hematoxylin.

34. The method of claim 32, wherein the staining comprises the use of a detectable label selected from the group consisting of a radioisotope, a fluorophore, a chemiluminescent compound, a bioluminescent compound, and a combination thereof.

35. The method of any one of claims 1-34, wherein the method further comprises imaging the biological sample.

36. The method of any one of claims 1-35, wherein the method further comprises before step (e), performing a step of extending a 3′ end of the extension product of step (d) using the capture probe as a template, thereby generating an extended capture product, and/or performing a step of extending a 3′ end of the capture probe using the extension product of step (d) as a template, thereby generating an extended capture probe.

37. The method of claim 36, wherein the extended capture product is removed from the capture probe on the array.

38. The method of claim 37, wherein the removing comprises use of heat or KOH.

39. The method of any one of claims 1-38, wherein the method further comprises generating a sequence library.

40. The method of any one of claims 1-39, wherein the determining in step (e) comprises sequencing.

41. The method of claim 40, wherein the sequencing comprises high-throughput sequencing.

42. The method of any one of claims 1-41, wherein the method further comprises a step of permeabilizing the biological sample.

43. The method of claim 42, wherein the permeabilizing comprises use of an organic solvent, a detergent, an enzyme, or a combination thereof.

44. The method of claim 42, wherein the permeabilizing comprises use of an endopeptidase, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

45. The method of claim 44, wherein the endopeptidase is pepsin or proteinase K.

46. The method of any one of claims 1-45, wherein the capture probe further comprises one or more functional domains, a unique molecular identifier, a cleavage domain, or combinations thereof.

47. The method of any one of claims 1-46, wherein the biological sample is a tissue sample.

48. The method of claim 47, wherein the tissue sample is a fixed tissue sample.

49. The method of claim 48, wherein the fixed tissue sample is a formalin-fixed paraffin embedded tissue sample, a paraformaldehyde fixed tissue sample, a methanol fixed tissue sample, or an acetone fixed tissue sample.

50. The method of claim 47, wherein the tissue sample is a fresh frozen tissue sample.

51. The method of any one of claims 1-47, wherein the biological sample is a tissue section.

52. The method of claim 51, wherein the tissue section is a fixed tissue section.

53. The method of claim 52, wherein the fixed tissue section is a formalin-fixed paraffin embedded tissue section, a paraformaldehyde fixed tissue section, a methanol fixed tissue section, or an acetone fixed tissue section.

54. The method of any one of claims 1-53, wherein extending the primer to generate an extension product in step (b) comprises use of a reverse transcriptase, optionally wherein the reverse transcriptase has strand displacement activity.

55. The method of any one of claims 1-54, wherein step (b) comprises generating a plurality of extension products using a plurality of primers.

56. The method of claim 55, wherein two or more primers of the plurality of primers hybridize to different sequences in the target nucleic acid.

57. The method of any one of claims 1-56, wherein the target nucleic acid encodes V and J sequences of an immune cell receptor.

58. The method of any one of claims 1-56, wherein the target nucleic acid encodes V, D, and J sequences of an immune cell receptor.

59. A method for determining a location of a target nucleic acid in a biological sample, the method comprising:
(a) contacting the biological sample with a plurality of primers, wherein the plurality of primers collectively comprise nucleic acid sequences that hybridize to complementary sequences in the target nucleic acid and a functional domain;
(b) hybridizing the plurality of primers to the target nucleic acid and extending one or more primers of the plurality of primers using the target nucleic acid as a template to generate one or more extension products;
(c) incorporating a polynucleotide sequence to the 3′ end of the one or more extension products;
(d) hybridizing the polynucleotide sequence of the one or more extension products of (c) to a plurality of capture probes on an array, wherein each capture probe in the plurality of capture probes comprises a spatial barcode and a capture domain; and
for an extension product in the one or more extension products, (e) determining (i) the sequence of the spatial barcode of the capture probe to which the extension product is hybridized, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

60. The method of claim 59, wherein the biological sample is disposed on the array comprising the plurality of capture probes.

61. The method of claim 59, wherein the biological sample is disposed on a substrate, preferably wherein the substrate does not comprise the array comprising the plurality of capture probes.

62. The method of claim 61, wherein the method further comprises aligning the substrate with the array, such that at least a portion of the biological sample is aligned with at least a portion of the array.

63. The method of claim 61 or 62, wherein the hybridizing in step (d) is facilitated by passive migration of the one or more extension products to the array.

64. The method of claim 61 or 62, wherein the hybridizing in step (d) is facilitated by active migration of the one or more extension products to the array, and optionally, wherein the active migration comprises electrophoresis.

65. The method of any one of claims 59-64, wherein the polynucleotide sequence in step (c) comprises a homopolynucleotide sequence, optionally 5′-CCC-3′.

66. The method of any one of claims 59-64, wherein the polynucleotide sequence in step (c) comprises a heteropolynucleotide sequence, optionally 5′-CGC-3′.

67. The method of any one of claims 59-66, wherein the capture domain comprises a fixed sequence.

68. The method of any one of claims 59-67, wherein a primer in the plurality of primers comprises a random sequence.

69. The method of claim 68, wherein the random sequence comprises a random hexamer.

70. The method of claim 68, wherein the random sequence comprises a random decamer.

71. The method of any one of claims 59-67, wherein the plurality of primers comprises a sequence complementary to a nucleic acid sequence encoding a constant region of an immune cell receptor.

72. The method of any one of claims 59-67, wherein the plurality of primers comprises a sequence complementary to a nucleic acid sequence encoding a constant region of a B cell receptor.

73. The method of any one of claims 59-67, wherein the plurality of primers comprises a sequence complementary to a nucleic acid sequence encoding a constant region of a T cell receptor.

74. The method of any one of claims 59-67, wherein a primer in the plurality of primers comprises a homopolymer sequence.

75. The method of claim 74, wherein the homopolymer sequence comprises a poly(T) sequence.

76. The method of any one of claims 59-75, wherein the array comprises one or more features.

77. The method of any one of claims 59-66, wherein the functional domain comprises a primer binding site or a sequencing specific site.

78. The method of any one of claims 59-77, wherein the target nucleic acid is an RNA.

79. The method of claim 78, wherein the RNA is mRNA.

80. The method of claim 79, wherein the mRNA comprises a sequence encoding a T cell receptor.

81. The method of claim 79, wherein the mRNA comprises a sequence encoding a B cell receptor.

82. The method of any one of claims 59-81, wherein incorporating the polynucleotide sequence to the 3′ end of the one or more extension products in step (c) comprises use of a reverse transcriptase.

83. The method of any one of claims 59-81, wherein incorporating the polynucleotide sequence to the 3′ end of the one or more extension products in step (c) comprises use of a terminal deoxynucleotidyl transferase.

84. The method of any one of claims 59-83, wherein the method further comprises removing the target nucleic acid, or any other nucleic acid hybridized to the one or more extension products, before the polynucleotide sequence hybridizes to the plurality of capture probes on the array.

85. The method of claim 84, wherein the removing comprises use of an RNase.

86. The method of claim 85, wherein the RNase is RNaseH.

87. The method of claim 84, wherein the removing comprises use of heat.

88. The method of any one of claims 59-87, wherein the method further comprises fixing the biological sample.

89. The method of claim 88, wherein fixing the biological sample comprises use of a fixative selected from the group consisting of: ethanol, methanol, acetone, formaldehyde, paraformaldehyde-Triton, glutaraldehyde, and combinations thereof.

90. The method of any one of claims 59-89, wherein the method further comprises staining the biological sample, optionally wherein the staining comprises use of eosin and/or hematoxylin.

91. The method of claim 90, wherein the staining comprises use of a detectable label selected from the group consisting of a radioisotope, a fluorophore, a chemiluminescent compound, a bioluminescent compound, and a combination thereof.

92. The method of any one of claims 59-91, wherein the method further comprises imaging the biological sample.

93. The method of any one of claims 59-92, wherein the method further comprises before step (e), performing a step of extending a 3′ end of the one or more extension products of step (d) using the capture probe as a template, thereby generating one or more extended capture products, and/or performing a step of extending a 3′ end of the capture probes using the one or more extension products of step (d) as a template, thereby generating one or more extended capture probes.

94. The method of claim 93, wherein the one or more extended capture products are removed from the capture probe on the array.

95. The method of claim 94, wherein the removing comprises use of heat or KOH.

96. The method of any one of claims 59-95, wherein the method further comprises generating a sequence library.

97. The method of any one of claims 59-96, wherein the determining in step (e) comprises sequencing.

98. The method of claim 97, wherein the sequencing comprises high-throughput sequencing.

99. The method of any one of claims 59-98, wherein the method further comprises a step of permeabilizing the biological sample.

100. The method of claim 99, wherein the permeabilizing comprises use of an organic solvent, a detergent, an enzyme, or a combination thereof.

101. The method of claim 99, wherein the permeabilizing comprises use of an endopeptidase, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

102. The method of claim 101, wherein the endopeptidase is pepsin or proteinase K.

103. The method of any one of claims 59-102, wherein the capture probe further comprises one or more functional domains, a unique molecular identifier, a cleavage domain, or combinations thereof.

104. The method of any one of claims 59-103, wherein the biological sample is a tissue sample.

105. The method of claim 104, wherein the tissue sample is a fixed tissue sample.

106. The method of claim 104, wherein the fixed tissue sample is a formalin-fixed paraffin embedded tissue sample, a paraformaldehyde fixed tissue sample, a methanol fixed tissue sample, or an acetone fixed tissue sample.

107. The method of claim 104, wherein the tissue sample is a fresh frozen tissue sample.

108. The method of any one of claims 59-103, wherein the biological sample is a tissue section.

109. The method of claim 108, wherein the tissue section is a fixed tissue section.

110. The method of claim 109, wherein the fixed tissue section is a formalin-fixed paraffin embedded tissue section, a paraformaldehyde fixed tissue section, a methanol fixed tissue section, or an acetone fixed tissue section.

111. The method of any one of claims 59-110, wherein the plurality of primers hybridize to different sequences in the target nucleic acid.

112. The method of claim 111, wherein two or more primers of the plurality of primers hybridize to different sequences in the target nucleic acid.

113. The method of claim 111 or 112, wherein ten or more primers of the plurality of primers hybridize to different sequences in the target nucleic acid.

114. The method of any one of claims 111-113, wherein fifty or more primers of the plurality of primers hybridize to different sequences in the target nucleic acid.

115. The method of any one of claims 111-114, wherein one hundred or more primers of the plurality of primers hybridize to different sequences in the target nucleic acid.

116. The method of any one of claims 59-115, wherein the extending in step (b) comprises use of a reverse transcriptase.

117. The method of claim 116, wherein the reverse transcriptase has strand displacement activity.

118. The method of claim 117, wherein the strand displacement activity of the reverse transcriptase displaces one or more primers of the plurality of primers from the target nucleic acid.

119. The method of claim 117 or 118, wherein the strand displacement activity of the reverse transcriptase displaces the one or more extension products from the target nucleic acid.

120. The method of any one of claims 59-115, wherein the extending in step (b) comprises the use of a reverse transcriptase and a helicase.

121. The method of claim 120, wherein the method further comprises use of one or more single-stranded DNA binding proteins.

122. The method of claim 121, wherein the one or more single-stranded DNA binding proteins comprises one or more of: Tth RecA, E. coli RecA, T4 gp32, and ET-SSB.

123. The method of any one of claims 120-122, wherein the helicase has strand displacement activity.

124. The method of any one of claims 59-115, wherein the extending in step (b) comprises the use of a superhelicase and a reverse transcriptase.

125. The method of claim 124, wherein the superhelicase is selected from the group consisting of: Rep, PrcA, UvrB, RecBCD, and Tte-Uvrd.

126. The method of claim 124 or 125, wherein the superhelicase has strand displacement activity.

127. The method of any one of claims 59-126, wherein the method further comprises generating two or more extension products from a primer of the plurality of primers.

128. The method of any one of claims 59-126, wherein the one or more extension products comprise different sequence lengths.

129. The method of any one of claims 59-128, wherein the functional sequence comprises a primer binding sequence or a sequencing specific sequence.

130. A kit comprising:
(a) an array comprising a plurality of capture probes, wherein a capture probe of the plurality of capture probes comprises:
(i) a spatial barcode; and
(ii) a capture domain that binds a capture sequence, or a complement thereof; and
(b) a plurality of primers, wherein a primer of the plurality of primers comprises a sequence complementary to a target nucleic acid and a functional domain, wherein the functional domain comprises a primer binding sequence or a sequencing specific sequence.

131. The kit of claim 130, wherein the capture probe further comprises one or more functional domains, a unique molecular identifier, a cleavage domain, or a combination thereof.

132. The kit of claim 130 or 131, wherein the kit further comprises a reverse transcriptase with strand displacement activity.

133. The kit of any one of claims 130-132, wherein the kit further comprises a polymerase.

134. The kit of any of any one of claims 130-133, wherein the capture domain comprises a fixed sequence.

135. The kit of any one of claims 130-134, wherein the primer of the plurality of primers comprises a sequence complementary to a nucleic acid encoding an immune cell receptor.

136. The kit of any one of claims 130-135, wherein the kit further comprises a helicase.

137. The kit of any one of claims 130-136, wherein the kit further comprises one or more single-stranded DNA binding proteins selected from the group comprising: Tth RecA, E. coli RecA, T4 gp32, and ET-SSB.

138. The kit of any one of claims, 130-137, wherein the kit further comprises a superhelicase, wherein the superhelicase is selected from the group consisting of: Rep, PrcA, UvrB, RecBCD, and Tte-Uvrd.

139. A composition comprising:
a) a target nucleic acid encoding a B cell receptor or a T cell receptor; and
b) one or more extension products hybridized to the target nucleic acid, wherein the one or more extension products each comprise in a 5′ to 3′ direction:
i) a primer hybridized to the target nucleic acid in a region encoding a constant region of the B cell receptor or T cell receptor, wherein the primer comprises a functional domain;
ii) a complementary sequence to the target nucleic acid, preferably wherein the complementary sequence is complementary to a sequence encoding V and J sequences of the B cell receptor or T cell receptor; and
iii) a capture sequence comprising a polynucleotide sequence.

140. The composition of claim 139, further comprising a reverse transcriptase or a terminal deoxynucleotidyl transferase.

141. The composition of claim 139 or 140, further comprising a helicase.

142. The composition of claim 141, further comprising a single-stranded DNA binding protein selected from the group comprising: Tth RecA, E. coli RecA, T4 gp32, and ET-SSB.

143. The composition of any one of claims 139-142, further comprising a superhelicase selected from the group consisting of: Rep, PrcA, UvrB, RecBCD, and Tte-Uvrd.

144. The composition of any one of claims 139-143, wherein the target nucleic acid is RNA.

145. The composition of claim 144, wherein the RNA is mRNA.

146. The composition of any one of claims 139-145, wherein the functional domain comprises a primer binding site or a sequencing specific site.

147. The composition of any one of claims 139-146, wherein the complementary sequence is further complementary to a sequence of the target nucleic acid encoding a D sequence.

148. The composition of any one of claims 139-147, wherein the polynucleotide sequence is a homopolynucleotide sequence, optionally wherein the homopolynucleotide sequence is 5′-CCC-3′.

149. The composition of any one of claims 139-147, wherein the polynucleotide sequence comprised in the capture sequence is a heteropolynucleotide sequence.

150. The composition of any one of claims 139-149, wherein the composition comprises two or more extension products hybridized to the target nucleic acid, wherein the two or more extension products each comprise a primer hybridized to a different region of the target nucleic acid.

151. A composition comprising:
one or more extension products wherein the one or more extension products comprise in a 5′ to 3′ direction comprising:
i) a primer, wherein the primer comprises a functional domain;
ii) a complementary sequence, wherein the complementary sequence is complementary to a target nucleic acid that encodes V and J sequences of an immune cell receptor; and
iii) a polynucleotide sequence,
wherein the polynucleotide sequence is hybridized to a capture domain on an array, wherein the array comprises a plurality of capture probes, wherein a capture probe of the plurality of capture probes comprises a spatial barcode and the capture domain, and wherein the capture domain comprises RNA.

152. The composition of claim 151, wherein the RNA comprises three or more ribonucleotides.

153. The composition of claim 151 or 152, wherein the capture probe further comprises a cleavage domain, one or more functional domains, a unique molecular identifier, or a combination thereof.

154. The method of any one of claims 151-153, wherein the functional domain comprises a primer binding sequence or a sequencing specific sequence.

155. The composition of any one of claims 151-154, wherein the target nucleic acid further encodes a D sequence of an immune cell receptor.

156. The composition of any one of claims 151-155, wherein the polynucleotide sequence comprises a homopolynucleotide sequence, optionally wherein the homopolynucleotide sequence is 5′-CCC-3′.

157. The composition of any one of claims 151-155, wherein the polynucleotide sequence comprises a heteropolynucleotide sequence.

158. A method for processing a target nucleic acid in a biological sample, the method comprising:
(a) contacting the biological sample with a primer comprising a nucleic acid sequence that hy bridizes to a complementary sequence in the target nucleic acid and a functional domain;
(b) hybridizing the primer to the target nucleic acid and extending the primer using the target nucleic acid as a template to generate an extension product;
(c) incorporating a polynucleotide sequence comprising at least three nucleotides to the 3′ end of the extension product; and
(d) hybridizing the polynucleotide sequence of the extension product to a capture domain on an array, wherein the array comprises a plurality of capture probes, and wherein a capture probe of the plurality of capture probes comprises a spatial barcode and the capture domain.

159. The method of claim 158, further comprising (e) determining (i) the sequence of the spatial barcode, or a complement thereof, and (ii) all or a portion of the sequence of the extension product corresponding to the target nucleic acid, or a complement thereof, and using the determined sequences of (i) and (ii) to determine the location of the target nucleic acid in the biological sample.

160. The method of any one of claims 1-129, 158, and 159, wherein the capture domain of the capture probe comprises a sequence that is complemenatry to the polynucleotide sequence, optionally wherein the sequence that is complemenatry to the polynucleotide sequence comprises RNA, preferably wherein the sequence that is complemenatry to the polynucleotide sequence comprises 5′-rGrGrG-3′or 5′-rGrCrG-3′.
